(19) United States
(12) Patent Application Publication
(10) Pub. No.: US 2026/0116847 A1
(43) Pub. Date: Apr. 30, 2026

(54) Crystalline polymorphic forms of marimastat, an amorphous form of marimastat, processes for their preparation, pharmaceutical compositions comprising the same, and uses thereof

(71) Applicant: PIKRALIDA SPÓLKA Z OGRANICZONA ODPOWIEDZIALNOSCIA, Poznan (PL)

(72) Inventors: Marek MASNYK, Warszawa (PL); Katarzyna SIDORYK, Warszawa (PL); Lukasz MUCHA, Warszawa (PL); Marta MAGDYCZ, Dabrowa Chotomowska (PL); Magdalena TYSZKIEWICZ, Warszawa (PL); Stanislaw PIKUL, Ceradz Dolny (PL)

(21) Appl. No.: 19/002,177

(22) Filed: Dec. 26, 2024

(30) Foreign Application Priority Data
Dec. 29, 2023 (PL) P.447396

(51) Int. Cl. C07C 237/22; A61K 31/16; C07C 231/12; C07C 231/24

(52) U.S. Cl. CPC C07C 237/22; A61K 31/16; C07C 231/12; C07C 231/24; C07B 2200/13

(57) ABSTRACT

The invention relates to a crystalline polymorphic form I, II and III of marimastat, a mixture of the crystalline polymorphic form I and III of marimastat, an amorphous form of marimastat, and processes for their preparation. The invention further relates to a pharmaceutical composition comprising the crystalline polymorphic form I, II or III of marimastat, the mixture of the crystalline polymorphic form I and III of marimastat, the amorphous form of marimastat or a mixture thereof. The invention also relates to the pharmaceutical composition for use as a medicament, and for use in the prevention and treatment of diseases associated with hyperactivity of extracellular matrix metalloproteinases. The invention further relates to the crystalline polymorphic form I, II or III of marimastat, the mixture of the crystalline polymorphic form I and III of marimastat, the amorphous form of marimastat or a mixture thereof for use as a medicament, and for use in the prevention and treatment of diseases associated with hyperactivity of extracellular matrix metalloproteinases.

### FIELD OF THE INVENTION

The present invention relates to crystalline polymorphic forms of marimastat, an amorphous form of marimastat, processes for their preparation, pharmaceutical compositions comprising the same, and their uses in the prevention and treatment of diseases associated with hyperactivity of extracellular matrix metalloproteinases.

### BACKGROUND OF THE INVENTION

Marimastat, or (2R,3S)—N-1-((S)-3,3-dimethyl-1-methylamino-1-oxobutan-2-yl)-N-4,3-dihydroxy-2-isobutylsuccinamide, is a compound with the structural formula:

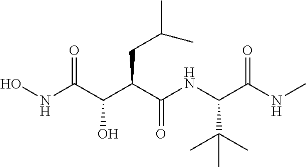 [SMILES: CNC(=O)[C@@H](NC(=O)[C@H](CC(C)C)[C@H](O)C(=O)NO)C(C)(C)C]

Marimastat is an inhibitor of extracellular matrix metalloproteinases (MMPs). Extracellular matrix metalloproteinases belong to a family of enzymes involved in the degradation of the extracellular matrix, which forms the tissue skeleton and allows the cell to exchange information with the environment. Excessive activity of extracellular matrix metalloproteinases causes destruction of the extracellular matrix, which results in improper functioning of the cell, which in turn contributes to the development of many diseases. Excessive activity of extracellular matrix metalloproteinases occurs, among others, in arthritis and periodontitis, atherosclerosis and cardiovascular diseases [1, 2, 3]. Metalloproteinases play an important role in the development and severity of vascular complications in diabetic patients [4]. They are believed to be involved in cell differentiation, migration and apoptosis, as well as in angiogenesis [3]. Metalloproteinases play an important role in carcinogenesis and metastasis. Their activity is correlated with the degree of malignancy of tumors and the tendency to metastasize [1, 2, 3]. Increased activity of extracellular matrix metalloproteinases is found in Alzheimer's disease, multiple sclerosis, amyotrophic lateral sclerosis, multiple myeloma and malignant melanoma [10, 11]. It has been proven that marimastat crosses the blood-brain barrier and has an inhibitory effect on the activity of metalloproteinases in the brain. In a mouse study, marimastat reduced the duration and number of seizures in a kainic acid-induced status epilepticus model [5]. Marimastat is also important in the treatment of advanced pancreatic cancer [6, 7, 8, 9]. Marimastat has been tested in over 400 patients in phase I/II trials in a wide variety of solid tumors. Most of these studies measured tumor-specific antigens (CEA in CRC, CA 19/9 in pancreatic cancer, CA 125 in ovarian cancer, prostate-specific antigen in prostate cancer) as surrogate markers of biological activity. A meta-analysis of all these studies, as well as analyzes of individual studies, showed that marimastat treatment significantly reduced all four growth indicators of tumor-specific antigens in a dose-dependent manner [9].

The beneficial effects of marimastat (PKL-021, FIG. 1) described in the literature cited above prompt more extensive research into both the use of marimastat in the treatment of a number of diseases and in-depth physicochemical studies of marimastat. A thorough literature analysis does not provide any information on physicochemical properties of marimastat, and there are only a few reports on its synthesis (PL 174279 B1; Richard J Davenport, Robert J Watson: “An improved synthesis of the broad spectrum matrix metalloprotease inhibitor marimastat”; Tetrahedron Letters, Volume 41, Issue 41, 7.10.2000, pp. 7983-7986; Kai Jenssen, Katherina Sewald and Norbert Sewald: “Synthesis of Marimastat and a Marimastat Conjugate for Affinity Chromatography and Surface Plasmon Resonance Studies”; Bioconjugate Chem. 2004, 15, 594-600; Daniel E. Levy, France Lapierre, Weisheng Liang, Wenqing Ye, Christopher W. Lange, Xiaoyuan Li, Damian Grobelny, Marie Casabonne, David Tyrrell, Kevin Holme, Alex Nadzan, and Richard E. Galardy: “Matrix Metalloproteinase Inhibitors: A Structure-Activity Study”; J. Med. Chem. 1998, 41, 199-223). Physicochemical properties of active substances used as drugs are extremely important during the development of the synthetic process itself, they play an important role in the process of preparing the appropriate formulation and, finally, they affect the rate of release, absorption and distribution in the body.

The development of a process for the synthesis of an active substance with favorable physicochemical parameters such as crystal size and its distribution, polymorphic form and its stability, bulk density, solubility are crucial in the course of filtration, washing and drying processes, and also affect the course of the medicinal product formulation process. During the entire technological process, active substances can be converted into various polymorphic forms that are more thermodynamically stable, but less soluble. It is also possible to observe the phenomenon of the formation of so-called pseudopolymorphs, i.e. solvates of substances with solvents, and hydrates, when the solvent incorporated into the crystal structure is water.

All the above physicochemical properties of the substance should be taken into account when planning to obtain a high-quality active substance and then a medicinal product with a specific release profile. A properly characterized active substance will enable technologists to develop the optimal formulation production process, select the appropriate equipment, as well as the type of granulation.

### SUMMARY OF THE INVENTION

The purpose of the invention is the preparation and complete physicochemical characterization of crystalline polymorphic forms of marimastat, an amorphous form of marimastat and the development of effective methods of their synthesis.

The subject of the invention is a crystalline polymorphic form of marimastat, which is the crystalline polymorphic form I, characterized by a powder X-ray diffraction pattern obtained by irradiation with Cu Kα radiation, which includes main peaks at 2-Theta° 6.11, 11.10, 12.19, 18.15, 19.57, 20.54, 20.74, 23.35, 24.50 and 26.20±0.2.

Preferably, the crystalline polymorphic form I is anhydrous.

Another subject of the invention is a crystalline polymorphic form of marimastat, which is the crystalline polymorphic form II, characterized by a powder X-ray diffraction pattern obtained by irradiation with Cu Kα radiation, which includes main peaks at 2-Theta° 6.18, 12.26, 14.95, 19.74, 21.53, 24.55, 24.55, 27.73, 30.80 and 37.12±0.2. Preferably, the crystalline polymorphic form II is anhydrous.

Another subject of the invention is a crystalline polymorphic form of marimastat, which is the crystalline polymorphic form III, characterized by a powder X-ray diffraction pattern obtained by irradiation with Cu Kα radiation, which includes main peaks at 2-Theta° 6.32, 11.05, 12.60, 17.30, 18.36, 20.43, 20.93, 24.05, 26.83 and 40.30±0.2. Preferably, the crystalline polymorphic form III is anhydrous.

Another subject of the invention is a mixture of the crystalline polymorphic form I of marimastat and the crystalline polymorphic form III of marimastat, characterized by a powder X-ray diffraction pattern obtained by irradiation with Cu Kα radiation, which includes main peaks at 2-Theta° 6.14, 6.30, 11.09, 12.22, 12.60, 18.43, 19.62, 20.51, 20.78 and 20.98±0.2.

Another subject of the invention is an amorphous form of marimastat.

Another subject of the invention is a process for the preparation of the crystalline polymorphic form I of marimastat according to the invention, characterized in that the process includes maceration of an amorphous form of marimastat in a solvent selected from the group comprising 2-methyltetrahydrofuran, isopropyl ether, methyl tert-butyl ether, methylene chloride, ethanol, cyclohexane, 1,2-dichloroethane, trichloroethylene, isopropyl acetate, 2-butanone, pentane and acetonitrile.

Preferably, the process includes the following steps: a) maceration of an amorphous form of marimastat in a solvent with stirring at room temperature for 2-4 days; and b) isolating the formed crystals, wherein the solvent is selected from the group comprising 2-methyltetrahydrofuran, isopropyl ether, methyl tert-butyl ether, methylene chloride, ethanol, cyclohexane, 1,2-dichloroethane, trichloroethylene, isopropyl acetate, 2-butanone, pentane and acetonitrile.

Another subject of the invention is a process for the preparation of the crystalline polymorphic form I of marimastat according to the invention, characterized in that the process includes preparing a saturated solution of marimastat in dimethyl sulfoxide and precipitating the crystals with acetonitrile, ethyl acetate or acetone.

Preferably, the process includes the following steps: a) preparing a saturated solution of marimastat in dimethyl sulfoxide at a temperature of 40-90° C.; b) optionally filtering the solution and washing with dimethyl sulfoxide; c) precipitating the crystals by adding acetonitrile, ethyl acetate or acetone to the solution and stirring the formed suspension at room temperature for 1-72 h; and d) isolating the formed crystals.

Another subject of the invention is a process for the preparation of the crystalline polymorphic form I of marimastat according to the invention, characterized in that the process includes crystallization from ethanol.

Preferably, the process includes the following steps: a) preparing a saturated solution of marimastat in ethanol 96% v/v or in anhydrous ethanol; b) stirring at room temperature for 5-7 days; and c) isolating the formed crystals.

Another subject of the invention is a process for the preparation of the crystalline polymorphic form II of marimastat according to the invention, characterized in that the process includes maceration of an amorphous form of marimastat in a solvent selected from chloroform or diethyl ether.

Preferably, the process includes the following steps: a) maceration of an amorphous form of marimastat in a solvent with stirring at room temperature for 2-5 days; and b) isolation of the formed crystals, wherein the solvent is selected from chloroform or diethyl ether.

Another subject of the invention is a process for the preparation of the crystalline polymorphic form II of marimastat according to the invention, characterized in that the process includes the following steps: a) preparing a saturated solution of marimastat in boiling methanol; b) adding 2-methyltetrahydrofuran, 1,2-dimethoxyethane or chloroform to the solution; c) cooling the solution to a temperature of 30-40° C.; and d) isolating the formed crystals.

Another subject of the invention is a process for the preparation of the crystalline polymorphic form II of marimastat according to the invention, characterized in that the process includes the following steps: a) preparing a saturated solution of marimastat in methanol at a temperature of 55-65° C.; b) mixing the solution with ethyl acetate at a temperature of 55-65° C.; c) cooling the mixture to room temperature; and d) isolating the formed crystals.

Another subject of the invention is a process for the preparation of the crystalline polymorphic form II of marimastat according to the invention, characterized in that the process includes the following steps: a) preparing a saturated solution of marimastat in dimethyl sulfoxide at a temperature of 40-90° C.; b) adding chloroform, methylene chloride, 2-methyltetrahydrofuran or 1,2-dimethoxyethane to the solution; c) cooling the solution to a temperature from room temperature to 40° C.; and d) isolating the formed crystals.

Another subject of the invention is a process for the preparation of the crystalline polymorphic form II of marimastat according to the invention, characterized in that the process includes the following steps: a) preparing a saturated solution of marimastat in dimethyl sulfoxide at a temperature of 50-65° C.; b) mixing the solution with ethyl acetate at a temperature of 55-65° C.; c) cooling the mixture to room temperature; and d) isolating the formed crystals.

Another subject of the invention is a process for the preparation of the crystalline polymorphic form II of marimastat according to the invention, characterized in that the process includes the following steps: a) reaction of (R)—N—((S)-3,3-dimethyl-1-methylamino-1-oxobutan-2-yl)-2-((S)-2,2-dimethyl-5-oxo-1,3-dioxolan-4-yl)-4-methylpentanamide with a 50% aqueous solution of hydroxylamine in 2-methyltetrahydrofuran or a mixture of dimethyl sulfoxide and chloroform at room temperature to boiling point; b) after the reaction is completed, performing azeotropic distillation to remove water; and c) isolating the formed crystals.

Another subject of the invention is a process for the preparation of the crystalline polymorphic form III of marimastat according to the invention, characterized in that the process includes preparing a saturated solution of marimastat in methanol, and precipitating the crystals with ethyl acetate.

Preferably, the process includes the following steps: a) preparing a saturated solution of marimastat in boiling methanol; b) cooling the solution to 50-60° C.; c) precipitating the crystals by adding ethyl acetate; d) stirring the suspension at boiling for 20-60 min. and azeotropic distillation of methanol; e) cooling the suspension to room temperature and stirring for 15-24 h; and f) isolating the formed crystals.

Another subject of the invention is a process for the preparation of the crystalline polymorphic form III of marimastat according to the invention, characterized in that the process includes preparing a saturated solution of marimastat in dimethyl sulfoxide and precipitating the crystals with a solution of methanol in water.

Preferably, the process includes the following steps: a) preparing a saturated solution of marimastat in dimethyl sulfoxide at a temperature of 60-75° C.; b) optionally filtering the solution; c) mixing the solution with a solution of methanol in water in a ratio of 1:7 v/v; d) allowing the solution to stand until crystals precipitate; and e) isolating the formed crystals.

Another subject of the invention is a process for the preparation of the mixture of the crystalline polymorphic form I of marimastat and the crystalline polymorphic form III of marimastat according to the invention, characterized in that the process includes maceration of an amorphous form of marimastat in a solvent selected from the group comprising tetrahydrofuran, 1,2-dimethoxyethane, 2-propanol, 1-propanol, isoamyl alcohol, acetone and heptane.

Preferably, the process includes the following steps: a) maceration of an amorphous form of marimastat in a solvent with stirring at room temperature for 2-5 days; and b) isolating the formed crystals, wherein the solvent is selected from the group comprising tetrahydrofuran, 1,2-dimethoxyethane, 2-propanol, 1-propanol, isoamyl alcohol, acetone and heptane.

Another subject of the invention is a process for the preparation of the mixture of the crystalline polymorphic form I of marimastat and the crystalline polymorphic form III of marimastat according to the invention, characterized in that the process includes preparing a saturated solution of marimastat in methanol and precipitating the crystals with acetonitrile, acetone, isopropyl acetate or 2-methyltetrahydrofuran.

Preferably, the process includes the following steps: a) preparing a saturated solution of marimastat in methanol at a temperature of 60-65° C.; b) mixing the solution with acetonitrile, isopropyl acetate or boiling acetone; c) stirring the formed suspension at room temperature for 15-24 h; and d) isolating the formed crystals.
Preferably, the process includes the following steps: a) preparing a saturated solution of marimastat in boiling methanol; b) cooling the solution to 50-60° C.; c) precipitating the crystals by adding 2-methyltetrahydrofuran; d) stirring the formed suspension at boiling for 20-60 min. and azeotropic distillation of methanol; e) cooling the suspension to room temperature and stirring for 15-24 h; and f) isolating the formed crystals.

Another subject of the invention is a process for the preparation of the mixture of the crystalline polymorphic form I of marimastat and the crystalline polymorphic form III of marimastat according to the invention, characterized in that the process includes crystallization from a boiling solvent selected from ethanol, n-propanol or isopropanol.

Preferably, the process includes the following steps: a) preparing a saturated solution of marimastat in a boiling solvent selected from ethanol, n-propanol or isopropanol; b) cooling the solution to room temperature and stirring for 15-24 h; and c) isolating the formed crystals.

Another subject of the invention is a process for the preparation of the mixture of the crystalline polymorphic form I of marimastat and the crystalline polymorphic form III of marimastat according to the invention, characterized in that the process includes preparing a saturated solution of marimastat in boiling ethanol and precipitating the crystals with ethyl acetate.

Preferably, the process includes the following steps: a) preparing a saturated solution of marimastat in boiling ethanol; b) precipitating the crystals by adding ethyl acetate; c) stirring the formed suspension at room temperature for 15-24 h; and d) isolating the formed crystals.

Another subject of the invention is a process for the preparation of the mixture of the crystalline polymorphic form I of marimastat and the crystalline polymorphic form III of marimastat according to the invention, characterized in that the process includes preparing a saturated solution of marimastat in dimethylformamide and precipitating the crystals with isopropyl acetate.

Preferably, the process includes the following steps: a) preparing a saturated solution of marimastat in dimethylformamide at a temperature of 70-85° C.; b) precipitating the crystals by adding isopropyl acetate; c) stirring the formed suspension at room temperature for 15-24 h; and d) isolating the formed crystals.

Another subject of the invention is a pharmaceutical composition characterized in that it comprises the crystalline polymorphic form I, II or III of marimastat according to the invention, the mixture of the crystalline polymorphic form I of marimastat and the crystalline polymorphic form III of marimastat according to the invention, the amorphous form of marimastat according to the invention or a mixture thereof and a pharmaceutically acceptable carrier.

The pharmaceutical composition according to the invention is administered by a route suitable for the type of drug form: enteral (e.g. orally, sublingually, rectally), parenteral (e.g. intravenously, intra-arterially, intramuscularly, subcutaneously, by inhalation), or topically (e.g. intraocularly, epidermally, vaginally).

The pharmaceutical composition according to the invention can be given a variety of pharmaceutical forms, well known to those skilled in the art, e.g. from Remington's Pharmaceutical Sciences, ed. 18, Mack Publ.Co.

Preferably, the pharmaceutical composition is in the form of a tablet, modified-release tablet, pill, capsule, powder, granules, pellets, suspension, emulsion, solution, oral liquid forms, e.g. syrup, solution or suspension for injection, solution for infusion, eye drops, ointment, gel, suppository, globule or therapeutic system, e.g. implant, vaginal ring, nanofiber. Pharmaceutical forms for oral administration include, for example, tablets, pills, powders, granules, pellets or capsules comprising solid pharmaceutically acceptable carriers such as corn starch, lactose, sucrose, sorbitol, talc, stearic acid, magnesium stearate, dicalcium phosphate or gums. Tablets or granules may be coated or otherwise processed to provide a dosage unit that provides the advantage of prolonged action.

A variety of substances can be used to form such protective or coating layers, including a variety of polymeric acids and mixtures of polymeric acids with substances such as shellac, ethyl alcohol or cellulose acetate.

Pharmaceutical forms for injection and infusion include sterile aqueous, aqueous-organic and non-aqueous solutions, suspensions, dry substances and tablets for solution or implantation. To prepare the suspension, excipients are used to ensure uniform dispersion of the medicinal substance in the liquid phase, such as polysorbates, lecithin, copolymers of polyoxyethylene with polyoxypropylene, peptizers such as phosphates, polyphosphates and citrates, water-soluble polymers such as carboxymethylcellulose, methylcellulose, polyvinylpyrrolidone, gums or gelatin. Preparations for injection may comprise pharmaceutically acceptable excipients such as pH adjusting and buffering agents, tonicifiers and preservatives. Dry substances are intended for preparing a solution or suspension ex tempore, by dilution with a suitable solvent.

Another subject of the invention is the pharmaceutical composition according to the invention for use as a medicament.

Another subject of the invention is the pharmaceutical composition according to the invention for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

Another subject of the invention is the crystalline polymorphic form I, II or III of marimastat according to the invention, the mixture of the crystalline polymorphic form I of marimastat and the crystalline polymorphic form III of marimastat according to the invention, the amorphous form of marimastat according to the invention or a mixture thereof for use as a medicament.

Another subject of the invention is the crystalline polymorphic form I, II or III of marimastat according to the invention, the mixture of the crystalline polymorphic form I of marimastat and the crystalline polymorphic form III of marimastat according to the invention, the amorphous form of marimastat according to the invention or a mixture thereof for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

The choice of drug dose and dosage regimen depends on the type of disease, age, weight and health of the patient and can be determined by a specialist based on known treatment regimens. In the treatment of post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications such as diabetic retinopathy and diabetic foot, the appropriate dose of the crystalline polymorphic form of marimastat according to the invention, the mixture of the crystalline polymorphic form I of marimastat and the crystalline polymorphic form III of marimastat according to the invention, the amorphous form of marimastat according to the invention or a mixture thereof may be from 0.05 to 50 mg/kg per day, preferably from 0.1 to 10 mg/kg per day. The appropriate dose may be administered to the patient once or several times a day, alone or in combination with other medicinal substances. Such substances can be administered simultaneously in the form of one preparation or separate preparations, or one after the other in an order and time interval determined by a specialist.

### BRIEF DESCRIPTION OF THE DRAWINGS

The subject of the invention is presented in more detail in the examples of implementation and in the drawing, in which

FIG. 1 shows the structural formula of marimastat (PKL-021);

FIG. 2 shows the XRPD pattern for the amorphous form;

FIG. 3 shows the XRPD pattern for the polymorphic form I;

FIG. 4 shows the XRPD pattern for the polymorphic form II;

FIG. 5 shows the XRPD pattern for the polymorphic form III;

FIG. 6 shows the XRPD pattern for the mixture of the polymorphic forms I and III;

FIG. 7 shows the thermogram from the TG analysis for the polymorphic form I, in which TGA is indicated by the top line, heat flow is indicated by the middle line, and the first derivative is indicated by the bottom line;

FIG. 8 shows the thermogram from the TG analysis for the polymorphic form II, in which TGA is indicated by the top line, heat flow is indicated by the middle line, and the first derivative is indicated by the bottom line;

FIG. 9 shows the thermogram from the TG analysis for the polymorphic form III, in which TGA is indicated by the top line, heat flow is indicated by the middle line, and the first derivative is indicated by the bottom line;

FIG. 10 shows a photo of the polymorphic form I from an optical microscope (polarized light, ×40 magnification);

FIG. 11 shows a photo of the polymorphic form II from an optical microscope (×400 magnification);

FIG. 12A shows a photo of the polymorphic form III from an electron microscope;

FIG. 12 B shows a photo of the polymorphic form III from an optical microscope (×100 magnification);

FIG. 13 shows solid-state ¹³C CPMAS NMR spectrum of the polymorphic form I;

FIG. 14 shows solid-state ¹³C CPMAS NMR spectrum of the polymorphic form II;

FIG. 15 shows solid-state ¹³C CPMAS NMR spectrum of the polymorphic form III;

FIG. 16 shows solid-state ¹³C CPMAS NMR spectrum of the mixture of the polymorphic forms I and III.

### DETAILED DESCRIPTION OF THE INVENTION

The obtained polymorphic forms of marimastat were characterized by X-ray powder diffraction (XRPD). Tables 1, 2, 3 and 4 contain the positions and intensities of the diffraction lines of the obtained forms I, II, III and the mixture of the forms I and III. Characteristic reflections used to identify the three polymorphic forms and the mixture of the forms I and III, as well as to determine the degree of their crystalline purity, are shown in bold in Tables 1, 2, 3 and 4.

Characteristic reflections of the form I occur at 2-Theta′ angles 6.11, 11.10, 12.19, 18.15, 19.57, 20.54, 20.74, 23.35, 24.50 and 26.20±0.2.

For the form II, characteristic reflections occur at 2-Theta′ angles 6.18, 12.26, 14.95, 19.74, 21.53, 24.55, 24.55, 27.73, 30.80 and 37.12±0.2.

For the form III, characteristic reflections occur at 2-Theta° angles 6.32, 11.05, 12.60, 17.30, 18.36, 20.43, 20.93, 24.05, 26.83 and 40.30±0.2.

The mixture of the forms I and III has characteristic reflections at 2-Theta′ angles 6.14, 6.30, 11.09, 12.22, 12.60, 18.43, 19.62, 20.51, 20.78, 20.98±0.2.

TABLE 1

| | | d-spacing | | Height |
|---|---|---|---|---|
| No. | Pos. [°2Th.] | [Å] | Rel. Int. [%] | [cts] |
| 1 | 5.52 | 16.01034 | 0.74 | 208.76 |
| 2 | 6.11 | 14.46557 | 100 | 28382.81 |
| 3 | 9.7879 | 9.03666 | 0.22 | 61.85 |
| 4 | 11.1006 | 7.97083 | 4.99 | 1417.69 |
| 5 | 12.1915 | 7.25998 | 14.91 | 4230.67 |
| 6 | 13.0289 | 6.79514 | 0.31 | 86.68 |
| 7 | 13.756 | 6.43757 | 0.65 | 185.23 |
| 8 | 14.751 | 6.00551 | 0.59 | 166.58 |
| 9 | 15.3261 | 5.78143 | 0.62 | 174.96 |
| 10 | 16.2554 | 5.45294 | 0.85 | 242.59 |
| 11 | 16.6079 | 5.33799 | 0.82 | 232.96 |
| 12 | 17.4328 | 5.08722 | 2.2 | 624.94 |
| 13 | 18.151 | 4.88752 | 2.38 | 675.34 |
| 14 | 18.5885 | 4.77345 | 1.69 | 480.03 |
| 15 | 19.5737 | 4.53537 | 4.41 | 1250.42 |
| 16 | 20.5445 | 4.3232 | 5.64 | 1601.87 |
| 17 | 20.735 | 4.28391 | 5.74 | 1630 |
| 18 | 22.3964 | 3.96974 | 1.46 | 414.2 |
| 19 | 23.3451 | 3.81052 | 3.14 | 892.06 |
| 20 | 23.7921 | 3.73993 | 0.61 | 174.47 |
| 21 | 24.1192 | 3.68995 | 0.82 | 231.79 |
| 22 | 24.5024 | 3.6331 | 3.35 | 950 |
| 23 | 25.3089 | 3.51913 | 0.91 | 257.92 |
| 24 | 26.204 | 3.40092 | 3.29 | 934.85 |
| 25 | 27.001 | 3.30231 | 1.19 | 337.9 |
| 26 | 27.6946 | 3.22116 | 0.3 | 85.29 |
| 27 | 28.0201 | 3.18448 | 0.46 | 129.67 |
| 28 | 28.4596 | 3.1363 | 0.56 | 157.76 |
| 29 | 29.1452 | 3.06406 | 0.69 | 196.71 |
| 30 | 29.5443 | 3.02357 | 1.67 | 473.03 |
| 31 | 30.724 | 2.91012 | 1.91 | 541.52 |
| 32 | 31.1201 | 2.87397 | 0.4 | 114.76 |
| 33 | 31.5892 | 2.83235 | 0.18 | 51.47 |
| 34 | 32.2013 | 2.7799 | 0.99 | 280 |
| 35 | 32.56 | 2.75009 | 0.19 | 54.45 |
| 36 | 33.55 | 2.67117 | 0.16 | 44.22 |
| 37 | 33.8282 | 2.64984 | 0.29 | 81.76 |
| 38 | 34.1948 | 2.62226 | 0.34 | 97.01 |
| 39 | 35.2536 | 2.54589 | 0.14 | 40.05 |
| 40 | 36.1814 | 2.48271 | 0.4 | 113.84 |
| 41 | 36.5888 | 2.456 | 0.33 | 92.59 |
| 42 | 37.061 | 2.42579 | 1.94 | 550 |
| 43 | 37.12 | 2.42206 | 1.55 | 440 |
| 44 | 37.8099 | 2.37944 | 0.4 | 113.78 |
| 45 | 38.09 | 2.36259 | 0.51 | 143.71 |
| 46 | 38.3193 | 2.34898 | 0.43 | 122.26 |
| 47 | 38.7083 | 2.32626 | 0.42 | 118.17 |
| 48 | 39.7 | 2.27041 | 0.34 | 96.17 |

TABLE 2

| | | d-spacing | | Height |
|---|---|---|---|---|
| No. | Pos. [°2Th.] | [Å] | Rel. Int. [%] | [cts] |
| 1 | 5.59 | 15.81001 | 0.82 | 456.97 |
| 2 | 6.1806 | 14.30049 | 100 | 56045.54 |
| 3 | 10.6465 | 8.30975 | 0.59 | 331.38 |
| 4 | 11.7602 | 7.52525 | 1.03 | 576.81 |
| 5 | 12.262 | 7.21835 | 15.5 | 8685.27 |
| 6 | 14.6356 | 6.05262 | 0.59 | 329.07 |
| 7 | 14.9538 | 5.92454 | 2.3 | 1291.61 |
| 8 | 16.34 | 5.42491 | 0.23 | 129.35 |
| 9 | 17.175 | 5.16299 | 1.25 | 700 |
| 10 | 17.6573 | 5.02303 | 0.54 | 304.69 |
| 11 | 17.9965 | 4.92912 | 0.99 | 552.98 |
| 12 | 18.4584 | 4.80682 | 1.27 | 711.66 |
| 13 | 18.7449 | 4.73399 | 0.63 | 353.42 |
| 14 | 19.0958 | 4.64777 | 0.82 | 461.17 |
| 15 | 19.7375 | 4.4981 | 1.61 | 903.43 |
| 16 | 20.3485 | 4.36438 | 1.28 | 720 |
| 17 | 21.534 | 4.12673 | 2.16 | 1209.88 |
| 18 | 22.5157 | 3.94897 | 0.31 | 171.1 |
| 19 | 23.4429 | 3.79485 | 0.4 | 225.89 |
| 20 | 23.7095 | 3.75277 | 0.58 | 323 |
| 21 | 24.1435 | 3.68629 | 0.53 | 299.71 |
| 22 | 24.5517 | 3.62592 | 3.41 | 1913.76 |
| 23 | 24.82 | 3.58733 | 0.89 | 500 |
| 24 | 25.0882 | 3.54958 | 1.17 | 653.56 |
| 25 | 25.3517 | 3.51328 | 2.17 | 1214.96 |
| 26 | 27.2553 | 3.27208 | 1.24 | 697.41 |
| 27 | 27.7291 | 3.21724 | 1.95 | 1095.27 |
| 28 | 28.5028 | 3.13164 | 1.19 | 664.42 |
| 29 | 29.422 | 3.03586 | 0.42 | 235.81 |
| 30 | 30.1363 | 2.96551 | 0.53 | 296.88 |
| 31 | 30.7962 | 2.90345 | 1.52 | 850.06 |
| 32 | 31.2366 | 2.86352 | 0.49 | 276.7 |
| 33 | 32.1333 | 2.78562 | 0.15 | 84.91 |
| 34 | 33.2472 | 2.6948 | 0.22 | 120.51 |
| 35 | 33.8769 | 2.64614 | 0.13 | 74.81 |
| 36 | 34.4835 | 2.60096 | 0.11 | 59.57 |
| 37 | 35.0383 | 2.56104 | 0.18 | 99.36 |
| 38 | 35.628 | 2.52 | 0.11 | 63.75 |
| 39 | 37.1247 | 2.42177 | 1.95 | 1094.19 |
| 40 | 37.9192 | 2.37283 | 0.12 | 64.96 |

TABLE 3

| | | d-spacing | | Height |
|---|---|---|---|---|
| No. | Pos. [°2Th.] | [Å] | Rel. Int. [%] | [cts] |
| 1 | 6.319 | 13.977 | 100.00 | 162 |
| 2 | 9.5679 | 9.2364 | 0.39 | 0.6 |
| 3 | 11.051 | 7.9998 | 17.27 | 31 |
| 4 | 12.600 | 7.0196 | 10.77 | 20 |
| 5 | 13.097 | 6.755 | 0.90 | 1.5 |
| 6 | 14.7386 | 6.0055 | 0.32 | 1.0 |
| 7 | 15.4742 | 5.7217 | 0.20 | 0.5 |
| 8 | 16.3800 | 5.4072 | 0.65 | 1.6 |
| 9 | 17.3017 | 5.1212 | 1.77 | 5 |
| 10 | 18.363 | 4.8275 | 16.81 | 30 |
| 11 | 19.1788 | 4.6240 | 1.24 | 2.7 |
| 12 | 19.563 | 4.5341 | 0.39 | 0.9 |
| 13 | 20.4277 | 4.34405 | 7.90 | 16 |
| 14 | 20.6946 | 4.2886 | 0.58 | 1.8 |
| 15 | 20.9324 | 4.24042 | 3.76 | 8 |
| 16 | 22.0638 | 4.0255 | 0.26 | 0.7 |
| 17 | 22.5449 | 3.94065 | 0.43 | 1.3 |
| 18 | 23.4045 | 3.7978 | 0.29 | 0.9 |
| 19 | 24.046 | 3.6980 | 3.24 | 6 |
| 20 | 25.2237 | 3.52789 | 0.92 | 2.2 |
| 21 | 25.846 | 3.4444 | 0.13 | 0.5 |
| 22 | 26.830 | 3.3202 | 2.64 | 6 |
| 23 | 27.402 | 3.2521 | 1.01 | 2.3 |
| 24 | 28.621 | 3.1164 | 0.95 | 1.6 |
| 25 | 30.0583 | 2.97056 | 0.36 | 0.9 |
| 26 | 31.1192 | 2.87167 | 0.21 | 0.7 |
| 27 | 31.6936 | 2.82092 | 0.61 | 1.6 |
| 28 | 32.942 | 2.7168 | 0.21 | 0.2 |
| 29 | 33.9107 | 2.64138 | 0.20 | 0.4 |
| 30 | 34.3929 | 2.60545 | 0.06 | 0.2 |
| 31 | 36.024 | 2.4911 | 0.16 | 0.5 |
| 32 | 36.592 | 2.45373 | 0.23 | 0.5 |
| 33 | 38.2503 | 2.35110 | 1.29 | 1.0 |
| 34 | 39.285 | 2.29153 | 0.50 | 1.2 |
| 35 | 40.302 | 2.2360 | 2.01 | 0.4 |
| 36 | 42.459 | 2.1273 | 1.00 | 0.2 |
| 37 | 43.856 | 2.06269 | 0.23 | 0.3 |
| 38 | 44.872 | 2.01831 | 0.10 | 0.2 |
| 39 | 45.885 | 1.97612 | 0.52 | 0.2 |

TABLE 4

| | | d-spacing | | Height |
|---|---|---|---|---|
| No. | Pos. [°2Th.] | [Å] | Rel. Int. [%] | [cts] |
| 1 | 6.1372 | 14.40162 | 100 | 23200 |
| 2 | 6.3048 | 14.01911 | 46.98 | 10900 |
| 3 | 9.8151 | 9.01174 | 0.34 | 77.84 |
| 4 | 11.0932 | 7.97618 | 9.7 | 2250 |
| 5 | 12.2194 | 7.24342 | 14.93 | 3463.43 |
| 6 | 12.5984 | 7.02639 | 7.99 | 1853.6 |
| 7 | 13.0962 | 6.76039 | 0.72 | 166.39 |
| 8 | 13.7812 | 6.42585 | 0.88 | 203.96 |
| 9 | 14.7582 | 6.00258 | 0.95 | 221 |
| 10 | 15.3587 | 5.76925 | 0.63 | 146.18 |
| 11 | 16.2942 | 5.44006 | 1.2 | 277.38 |
| 12 | 16.6486 | 5.32503 | 0.92 | 214.02 |
| 13 | 17.3745 | 5.10416 | 2.72 | 630.83 |
| 14 | 18.1526 | 4.88708 | 2.99 | 694.09 |
| 15 | 18.4254 | 4.81535 | 8.21 | 1904.17 |
| 16 | 18.6257 | 4.76402 | 3.23 | 750 |
| 17 | 19.2259 | 4.61662 | 0.99 | 230.14 |
| 18 | 19.6188 | 4.52505 | 6.08 | 1409.52 |
| 19 | 20.513 | 4.32618 | 7.76 | 1800 |
| 20 | 20.7778 | 4.27163 | 7.76 | 1800 |
| 21 | 20.9843 | 4.23357 | 4.09 | 950 |
| 22 | 22.3778 | 3.96971 | 1.2 | 278.63 |
| 23 | 23.3823 | 3.8014 | 2.87 | 665.75 |
| 24 | 24.0855 | 3.69197 | 3.01 | 697.67 |
| 25 | 24.5085 | 3.62921 | 3.38 | 784.31 |
| 26 | 25.28 | 3.52017 | 1.66 | 384.11 |
| 27 | 26.2328 | 3.39443 | 3.23 | 748.7 |
| 28 | 26.9049 | 3.31114 | 3.24 | 750.79 |
| 29 | 27.4331 | 3.24858 | 1.33 | 308.1 |
| 30 | 28.0482 | 3.17872 | 0.4 | 91.85 |
| 31 | 28.5074 | 3.12855 | 0.84 | 194.17 |
| 32 | 28.7224 | 3.10562 | 1.04 | 242.14 |
| 33 | 29.1971 | 3.0562 | 0.65 | 151.18 |
| 34 | 29.5619 | 3.01931 | 1.44 | 334.57 |
| 35 | 30.1391 | 2.96279 | 0.49 | 112.63 |
| 36 | 30.7761 | 2.9029 | 1.76 | 408.58 |
| 37 | 31.1751 | 2.86665 | 0.66 | 153.32 |
| 38 | 31.7026 | 2.82014 | 0.84 | 194.46 |
| 39 | 32.229 | 2.77528 | 0.81 | 188.17 |
| 40 | 32.54 | 2.75173 | 0.31 | 71.92 |
| 41 | 33.0326 | 2.70957 | 0.17 | 39.58 |
| 42 | 33.88 | 2.6459 | 0.33 | 76.49 |
| 43 | 34.2186 | 2.61832 | 0.33 | 77.09 |
| 44 | 36.2354 | 2.47708 | 0.29 | 67.92 |
| 45 | 36.6473 | 2.45018 | 0.35 | 81.92 |
| 46 | 37.0966 | 2.42153 | 1.75 | 405.03 |
| 47 | 37.919 | 2.37088 | 0.75 | 173.69 |
| 48 | 38.27 | 2.35189 | 1.28 | 296.87 |
| 49 | 38.771 | 2.32072 | 0.22 | 51.53 |
| 50 | 39.3555 | 2.28759 | 0.31 | 70.84 |
| 51 | 39.77 | 2.26657 | 0.36 | 84.47 |

### Example 1

### Preparation of the Amorphous Form

2 g of marimastat was dissolved in water (500 mL) at room temperature. The clear solution was frozen in acetone/dry ice bath and lyophilized for 48 hours. The amorphous material was obtained in quantitative yield.

### Example 2

### Preparation of the Amorphous Form

50 mg of marimastat was ground at 30 Hz for 1.5-3 hours in a ball mill. The amorphous material was obtained in quantitative yield.

### Example 3

### Preparation of the Crystalline Polymorphic Form I

An amorphous form was suspended in a solvent selected from the group: 2-methyltetrahydrofuran (2-MeTHF), isopropyl ether, methyl tert-butyl ether (MTBE), methylene chloride, ethanol (EtOH), cyclohexane, 1,2-dichloroethane, trichloroethylene, isopropyl acetate, 2-butanone, pentane and acetonitrile, and then stirred at room temperature for 2-4 days. The white suspension was placed in Eppendorf. The polymorphic form I was centrifuged, dried at room temperature and under high vacuum.

### Example 4

### Preparation of the Crystalline Polymorphic Form I

Marimastat was dissolved in dimethyl sulfoxide (DMSO) at a temperature of 50° C., filtered through Schott and washed with DMSO. Then, acetonitrile was added to the clear solution, and the formed suspension was stirred at room temperature for 3 days. The crystals were filtered off, washed with acetonitrile and dried at room temperature and under high vacuum to obtain the form I with a yield of 70%.

### Example 5

### Preparation of the Crystalline Polymorphic Form I

Marimastat was heated in anhydrous ethanol or 96% v/v ethanol until dissolved and then stirred at room temperature for 7 days. The crystals were filtered off, washed with anhydrous ethanol or ethanol 96% v/v, dried at room temperature and under high vacuum to obtain the form I with yields of 75% and 65%, respectively.

### Example 6

### Preparation of the Crystalline Polymorphic Form I

Marimastat was dissolved in DMSO at a temperature of 50° C. Then, ethyl acetate was added to the clear solution, and the formed suspension was stirred for 2 hours at room temperature. The crystals were filtered off, washed with ethyl acetate, dried at room temperature and under high vacuum to obtain the form I with a yield of 60%.

### Example 7

### Preparation of the Crystalline Polymorphic Form I

Marimastat was dissolved in DMSO at a temperature of 50° C. Then, acetone was added to the clear solution and the formed suspension was stirred for 2 hours at room temperature. The crystals were filtered off, washed with acetone, dried at room temperature and under high vacuum to obtain the form I with a yield of 46%.

### Example 8

### Preparation of the Crystalline Polymorphic Form II

An amorphous form was suspended in chloroform or diethyl ether and stirred at room temperature for 2-4 days. The white suspension was placed in Eppendorf. The polymorphic form II was centrifuged, dried at room temperature and under high vacuum.

### Example 9

### Preparation of the Crystalline Polymorphic Form II

Marimastat was dissolved in boiling methanol (MeOH), and 2-methyltetrahydrofuran (2-MeTHF) was added. After cooling to room temperature, the formed suspension was stirred for 24 hours. The crystals were filtered off, washed with 2-MeTHF and dried at room temperature and under high vacuum to obtain the form II with a yield of 60%.

### Example 10

### Preparation of the Crystalline Polymorphic Form II

Marimastat was dissolved in boiling methanol (MeOH), and 2-methyltetrahydrofuran (2-MeTHF) was added. After cooling to a temperature of 40° C., the crystals were filtered off, washed with 2-MeTHF and dried at room temperature and under high vacuum to obtain the form II with a yield of 48%.

### Example 11

### Preparation of the Crystalline Polymorphic Form II

Marimastat was dissolved in boiling methanol (MeOH), and 1,2-dimethoxyethane was added. After cooling to 40° C., the crystals were filtered off, washed with 1,2-dimethoxyethane and dried at room temperature and under high vacuum to obtain the form II with a yield of 61%.

### Example 12

### Preparation of the Crystalline Polymorphic Form II

Marimastat was dissolved in boiling methanol (MeOH), and chloroform was added. After cooling to 40° C., the crystals were filtered off, washed with chloroform and dried at room temperature and under high vacuum to obtain the form II with a yield of 40%.

### Example 13

### Preparation of the Crystalline Polymorphic Form II

Marimastat was dissolved in dimethyl sulfoxide (DMSO) at a temperature of 50° C. and chloroform was added. After cooling to 40° C., the crystals were filtered off, washed with chloroform and dried at room temperature and under high vacuum to obtain the form II with a yield of 89%.

### Example 14

### Preparation of the Crystalline Polymorphic Form II

Marimastat was dissolved in methanol (MeOH) at a temperature of 60° C., and the solution was added to ethyl acetate (AcOEt) at a temperature of 60° C. After cooling to room temperature, the crystals were filtered off, washed with AcOEt, dried at room temperature and under high vacuum to obtain the form II with a yield of 83%.

### Example 15

### Preparation of the Crystalline Polymorphic Form II

Marimastat was dissolved in dimethyl sulfoxide (DMSO) at a temperature of 60° C. and the solution was added to ethyl acetate (AcOEt) at a temperature of 60° C. After cooling to room temperature, the crystals were filtered off, washed with AcOEt, dried at room temperature and under high vacuum to obtain the form II with a yield of 55%.

### Example 16

### Preparation of the Crystalline Polymorphic Form II

Marimastat was dissolved in dimethyl sulfoxide (DMSO) at a temperature of 50° C. and methylene chloride was added. After cooling to a room temperature, the crystals were filtered off, washed with methylene chloride, dried at room temperature and under high vacuum to obtain the form II with a yield of 90%.

### Example 17

### Preparation of the Crystalline Polymorphic Form II

(R)—N—((S)-3,3-dimethyl-1-methylamino-1-oxobutan-2-yl)-2-((S)-2,2-dimethyl-5-oxo-1,3-dioxolan-4-yl)-4-methylpentanamide (45 g) (obtained by the process disclosed in Tetrahedron Letters-[12]) was poured with 2-methyltetrahydrofuran (2-MeTHF, 900 mL) and heated to a temperature of 40-45° C. to obtain a clear solution. Then, a 50% aqueous solution of hydroxylamine (50% NH₂OH, 29 mL) was added and the reaction was carried out for 1-2 hours at a temperature of 40-45° C. After the reaction was completed, acetone (90 mL) was added and the reaction mixture was stirred at boiling for 20-30 min. This was followed by azeotropic distillation to remove water and, once completed, the suspension was stirred for 15-20 hours at room temperature. The crystals were filtered off, washed with 2-MeTHF and dried at room temperature to obtain the form II with a yield of 88% (37 g).

### Example 18

### Preparation of the Crystalline Polymorphic Form II

(R)—N—((S)-3,3-dimethyl-1-methylamino-1-oxobutan-2-yl)-2-((S)-2,2-dimethyl-5-oxo-1,3-dioxolan-4-yl)-4-methylpentanamide (10 g, 28 mmol, 1 eq) (obtained by the process disclosed in Tetrahedron Letters-[12]) was dissolved in 14 mL of dimethyl sulfoxide (DMSO) and 14 mL of chloroform at room temperature. Then, NH₂OH (5.2 mL, 50% aqueous solution, 84 mmol, 3.0 eq) was added. The suspension was stirred at room temperature for 1 h (TLC monitoring: 10% CH₃OH/CH₂Cl₂). Then, 9 mL of acetone was added dropwise. The reaction mixture was stirred at boiling for 30 minutes. Then, the reaction mixture was subjected to azeotropic distillation from the mixture of DMSO, chloroform and acetone to remove water. The volume of distilled solvent was replenished by successive additions of further portions of chloroform. Then, the entire volume of chloroform was distilled off and 185 mL of chloroform was added dropwise to the obtained clear solution of marimastat in DMSO while maintaining boiling. Then, the reaction mixture was cooled to room temperature and stirred for 17 hours. The crystals were filtered off, washed with chloroform (15 mL), acetone (3×15 mL), and dried to obtain the form II with a yield of 83% (7.71 g).

### Example 19

### Preparation of the Crystalline Polymorphic Form III

A marimastat suspension (202 g) was stirred in boiling methanol (1.2 L) until dissolved. Then, the clear solution was cooled to 60° C. and ethyl acetate (2 L) was added. The suspension was stirred at boiling for 20 min, and then methanol was removed by azeotropic distillation. The suspension was cooled to room temperature and stirred for 24 hours. The crystals were filtered off, washed with ethyl acetate and dried at room temperature to obtain 192 g of the form III with a yield of 95%.

### Example 20

### Preparation of the Crystalline Polymorphic Form III

Marimastat was dissolved in DMSO at a temperature of 72° C. The solution was filtered through Schott and washed with DMSO. The clear solution was added to H₂O/MeOH (7:1 v/v) mixture and the formed suspension was stirred for 24 hours at room temperature. The crystals were filtered off, washed with H₂O and MeOH and dried at room temperature and under high vacuum to obtain the form III with a yield of 60%.

### Example 21

### Preparation of the Mixture of the Crystalline Polymorphic Forms I and III

An amorphous form was suspended in a solvent selected from the group: tetrahydrofuran, 1,2-dimethoxyethane, 2-propanol, 1-propanol, isoamyl alcohol, acetone and heptane, and then stirred at room temperature for 4 days. The white suspension was placed in Eppendorf. The mixture of the forms I and III was centrifuged, dried at room temperature and under high vacuum.

### Example 22

### Preparation of the Mixture of the Crystalline Polymorphic Forms I and III

Marimastat was dissolved in MeOH at a temperature of 65° C., and the clear solution was added to acetonitrile at room temperature. The formed suspension was stirred for 24 hours at room temperature. The crystals were filtered off, washed with acetonitrile, dried at room temperature and under high vacuum to obtain the mixture of the forms I and III with a yield of 80%.

### Example 23

### Preparation of the Mixture of the Crystalline Polymorphic Forms I and III

Marimastat was dissolved in MeOH at a temperature of 65° C., and the clear solution was added to boiling acetone. The formed suspension was stirred for 24 hours at room temperature. The crystals were filtered off, washed with acetone, dried at room temperature and under high vacuum to obtain the mixture of the forms I and III with a yield of 60%.

### Example 24

### Preparation of the Mixture of the Crystalline Polymorphic Forms I and III

Marimastat was dissolved in MeOH at boiling and then isopropyl acetate was added. The formed suspension was stirred for 24 hours at room temperature. The crystals were filtered off, washed with isopropyl acetate, dried at room temperature and under high vacuum to obtain the mixture of the forms I and III with a yield of 89%.

### Example 25

### Preparation of the Mixture of the Crystalline Polymorphic Forms I and III

Marimastat was suspended in n-propanol and stirred at boiling until dissolved. The clear solution was cooled to room temperature and stirred for 24 hours. The crystals were filtered off, washed with n-propanol, dried at room temperature and under high vacuum to obtain the mixture of the forms I and III with a yield of 83%.

### Example 26

### Preparation of the Mixture of the Crystalline Polymorphic Forms I and III

Marimastat was dissolved in boiling anhydrous ethanol (EtOH). The clear solution was cooled to room temperature and stirred for 24 hours. The crystals were filtered off, washed with EtOH and dried at room temperature and under high vacuum to obtain the mixture of the forms I and III with a yield of 67%.

### Example 27

### Preparation of the Mixture of the Crystalline Polymorphic Forms I and III

Marimastat was suspended in isopropanol and stirred at boiling until dissolved. The clear solution was cooled to room temperature and stirred for 24 hours. The crystals were filtered off, washed with isopropanol, dried at room temperature and under high vacuum to obtain the mixture of the forms I and III with a yield of 70%.

### Example 28

### Preparation of the Mixture of the Crystalline Polymorphic Forms I and III

Marimastat was dissolved in ethanol at boiling and then ethyl acetate was added. The formed suspension was stirred for 24 hours at room temperature. The crystals were filtered off, washed with ethyl acetate, dried at room temperature and under high vacuum to obtain the mixture of the forms I and III with a yield of 77%.

### Example 29

### Preparation of the Mixture of the Crystalline Polymorphic Forms I and III

Marimastat was dissolved in dimethylformamide (DMF) at a temperature of 85° C., and then isopropyl acetate was added dropwise. The formed suspension was stirred for 24 hours at room temperature. The crystals were filtered off, washed with isopropyl acetate, dried at room temperature and under high vacuum to obtain the mixture of the forms I and III with a yield of 95%.

### Example 30

### Preparation of the Mixture of the Crystalline Polymorphic Forms I and III

A marimastat suspension was stirred in boiling methanol until dissolved. Then, the clear solution was cooled to 60° C. and 2-methyltetrahydrofuran (2-MeTHF) was added. The suspension was stirred at boiling for 20 min, and then methanol was removed by azeotropic distillation. The suspension was cooled to room temperature and stirred for 24 hours. The crystals were filtered off, washed with 2-MeTHF and dried at room temperature and under high vacuum to obtain the mixture of the forms I and III with a yield of 98%.

### Example 31

### X-Ray Powder Diffraction (XRPD) Analysis

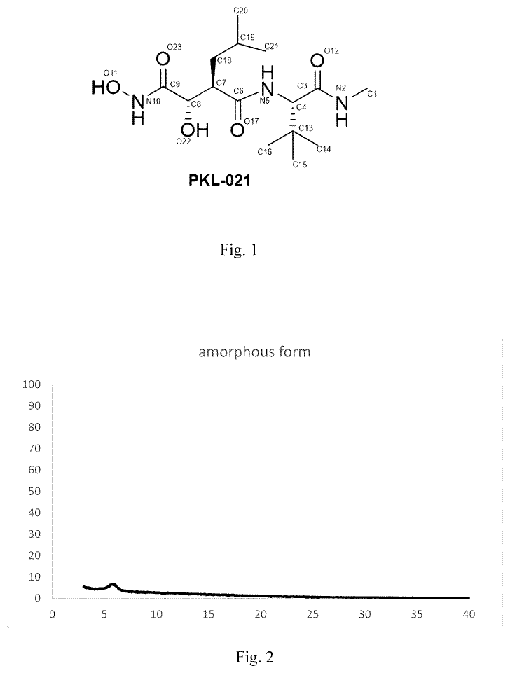

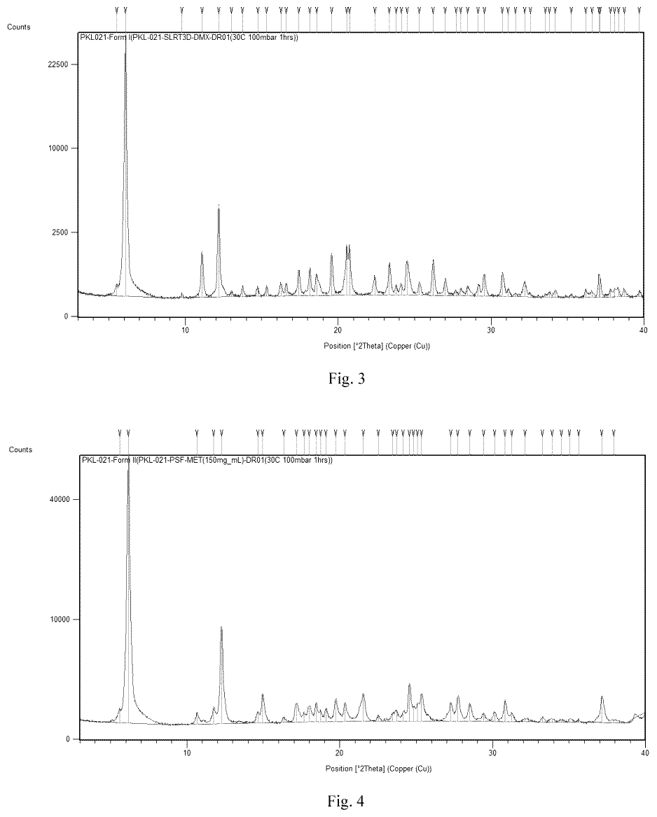

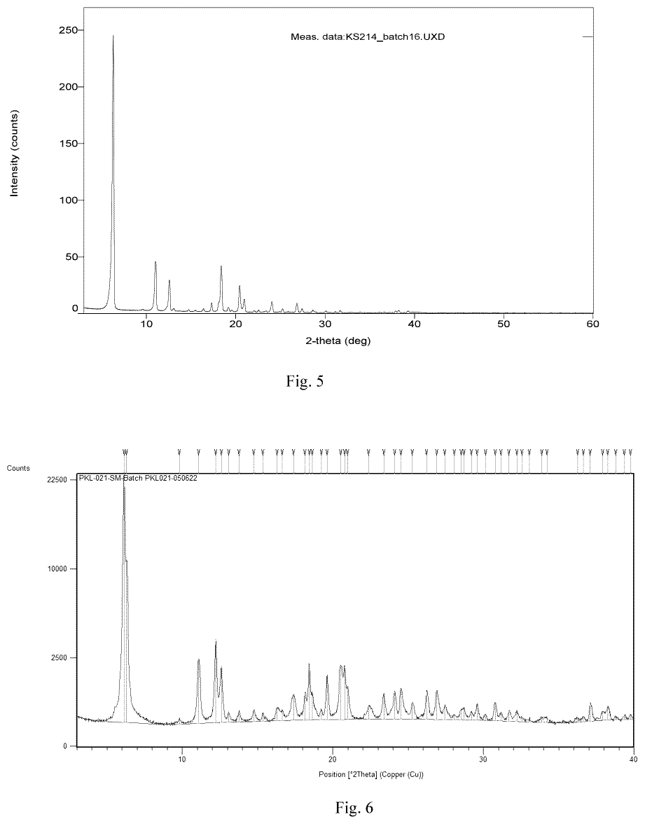

Powder diffractograms were collected using a Rigaku MiniFlex600 apparatus, radiation at Cu Kα1 wavelength, λ=1.54 Å for the crystalline polymorphic form I, II and the mixture of the forms I and III and using a Bruker D8 Advance apparatus, radiation at Cu Kα1 wavelength, Δ=1.54 Å for the crystalline polymorphic form III and the amorphous form. The XRPD patterns for the amorphous form, the crystalline polymorphic form I, II, III and the mixture of the forms I and III are shown in FIGS. 2, 3, 4, 5 and 6, respectively.

### Example 32

### Thermogravimetric Analysis (TGA)

Thermogravimetric analysis for the crystalline polymorphic form I, II and III was performed using a Mettler Toledo TGA/DSC1 apparatus. The sample was weighed in an aluminum pan closed with an aluminum cover with holes.

### The Crystalline Polymorphic Form I

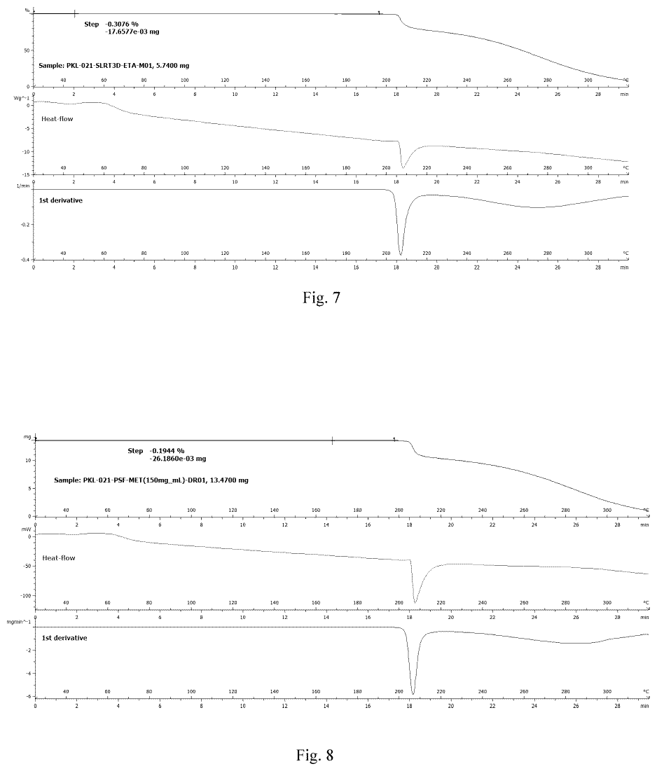

The TG analysis was performed in the range of 25° C.-320° C. at a rate of 10° C./min. The TG profile showed a weight loss of 0.3% at a temperature of 25° C.-195° C. The degradation occurred above 200° C. The TG analysis proves that the crystalline polymorphic form I of marimastat exists in the anhydrous form. The thermogram from the TG analysis for the crystalline polymorphic form I is shown in FIG. 7.

### The Crystalline Polymorphic Form II

The TG analysis was performed in the range of 25° C.-320° C. at a rate of 10° C./min. A weight loss of 0.2% was demonstrated at a temperature of 25° C.-195° C. The degradation occurred above 200° C. The TG analysis proves that the crystalline polymorphic form II of marimastat exists in the anhydrous form. The thermogram from the TG analysis for the crystalline polymorphic form II is shown in FIG. 8.

### The Crystalline Polymorphic Form III

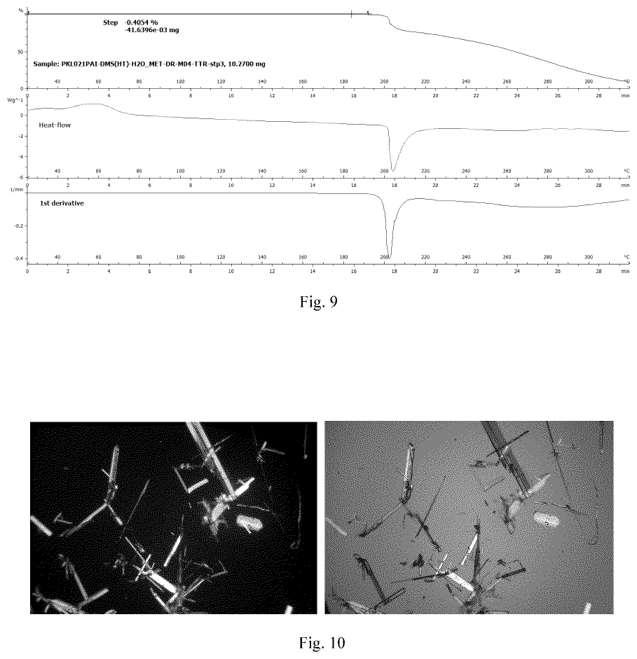

This sample was analyzed by TGA-EGA. The analysis was performed at a temperature of 25° C.-320° C. at a rate of 10° C./min. A weight loss of 0.4% was demonstrated at a temperature of 25° C.-195° C. The degradation occurred above 200° C. The TG analysis proves that the crystalline polymorphic form III of marimastat exists in the anhydrous form. The thermogram from the TG analysis for the crystalline polymorphic form III is shown in FIG. 9.

### CONCLUSIONS

Analysis of the results by X-ray powder diffraction and the thermogravimetric analysis indicates that three pure anhydrous crystalline polymorphic forms of marimastat and the mixture of the crystalline polymorphic forms I and III were obtained and characterized.

### Example 33

### Stability Studies

Stability studies of the amorphous form, pure crystalline polymorphic forms I, II and III and the mixture of the crystalline polymorphic forms I and III were carried out under the following conditions: 25° C./100% RH (relative humidity) and 40° C./75% RH (relative humidity) for 7 days, at 30°/100 mbar for 1 hour, at room temperature for 1 year, and also at elevated temperature (above 150° C.). The XRPD analyses of the tested samples did not show any changes in the image of diffraction lines, which proves that all three polymorphic forms, the mixture of the forms I and III and the amorphous form of marimastat are stable.

Stability studies of the obtained crystalline polymorphic forms of marimastat in various solvents were also carried out by stirring the forms I, II and III in an appropriate solvent at room temperature for 4 days. The XRPD analyses of the samples indicate that the most thermodynamically stable form is the form I (Table 5).

TABLE 5

| | | Conc. | Temp. | | |
|---|---|---|---|---|---|
| No. | solvent | [mg/mL] | [° C.] | Time | XRPD |
| 1 | acetonitrile | 10 | 25 | 4 d | Form I |
| 2 | acetone | 10 | 25 | 4 d | Form I |
| 3 | dimethoxyethane | 10 | 25 | 4 d | Form I |
| 4 | ethanol | 10 | 25 | 4 d | Form I |
| 5 | methanol | 110 | 25 | 4 d | Form I |
| 6 | tetrahydrofuran | 10 | 25 | 4 d | Form I |
| 7 | butyl acetate | 10 | 25 | 4 d | Form I + Form II + Form III |
| 8 | methylene chloride | 10 | 25 | 4 d | Form I + Form II + Form III |
| 9 | heptane | 10 | 25 | 4 d | Form I + Form II + Form III |
| 10 | methylcyclohexane | 10 | 25 | 4 d | Form I + Form II + Form III |
| 11 | methyl isobutyl ketone | 10 | 25 | 4 d | Form I + Form II + Form III |
| 12 | p-xylene | 10 | 25 | 4 d | Form I + Form II + Form III |
| 13 | toluene | 10 | 25 | 4 d | Form I + Form II + Form III |
| 14 | 2-propanol | 30 | 25 | 4 d | Form I + Form III |
| 15 | methyl tert-butyl ether | 10 | 25 | 4 d | Form I + Form III |
| 16 | water | 30 | 25 | 4 d | Form I + Form III |

The XRPD analysis of the samples showed the conversion of the mixture of three polymorphic forms into the pure form I in 6 solvents (items 1-6, Table 5), and in three solvents into the mixture of the forms I and III (items 14-16, Table 5). The remaining 7 solvents gave the initial mixture of three polymorphic forms. Thus, when using the mixture of all three forms for maceration in different solvents, complete disappearance of the form II and the form III, complete disappearance of the form II, or all three forms remaining in the mixture were observed. The complete disappearance of the form I was not observed in any of the experiments. The above experiments show that both the form III and the form II are kinetic forms, and the most thermodynamically stable is the form I of marimastat.

The stability of the form II in various solvents at room temperature was tested. Experiments were performed by suspending the form II in 1 ml of solvent and the formed suspension was stirred at room temperature at 25° C. for 4 days. Then, the precipitate was filtered off and the samples were dried at a temperature of 30° C./100 mbar for one hour, followed by XRPD analysis. The obtained results are presented in Table 6.

TABLE 6

| | | Conc. | | | |
|---|---|---|---|---|---|
| No. | Solvent | [mg/mL] | Temp. [° C.] | Time | XRPD |
| 1 | 2-propanol | 30 | 25 | 4 d | Form II |
| 2 | acetonitrile | 10 | 25 | 4 d | Form I |
| 3 | butyl acetate | 10 | 25 | 4 d | Form Il |
| 4 | methylene chloride | 20 | 25 | 4 d | Form II |
| 5 | ethanol | 10 | 25 | 4 d | Form II |
| 6 | water | 30 | 25 | 4 d | Form II |
| 7 | methylcyclohexane | 10 | 25 | 4 d | Form II |
| 8 | methanol | 110 | 25 | 4 d | Form I |
| 9 | p-xylene | 20 | 25 | 4 d | Form II |

The XRPD analysis of the samples showed complete conversion of the form II into the form I in two solvents: acetonitrile and methanol (items 2 and 8, Table 6). In other solvents, the form II is the stable form. Formation of the form III was not observed. The above experiment confirmed that the form I of marimastat has the greatest thermodynamic stability.

### Example 34

### Microscopic Examinations

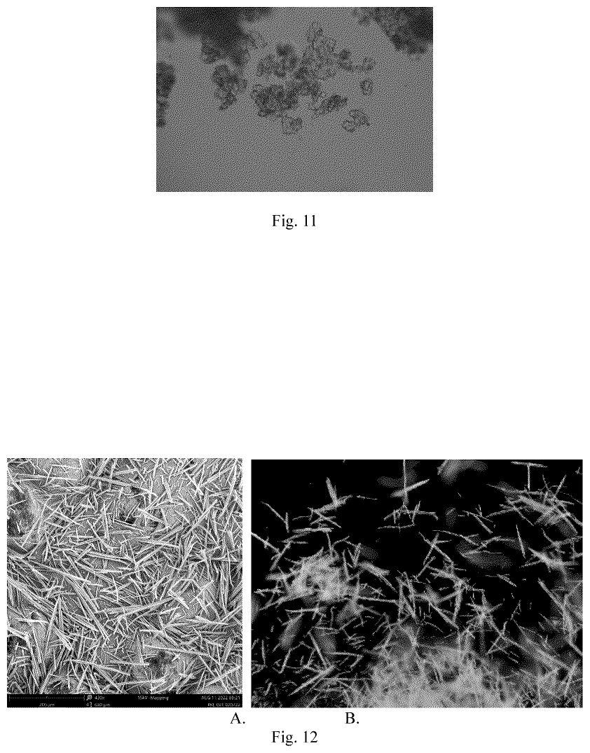

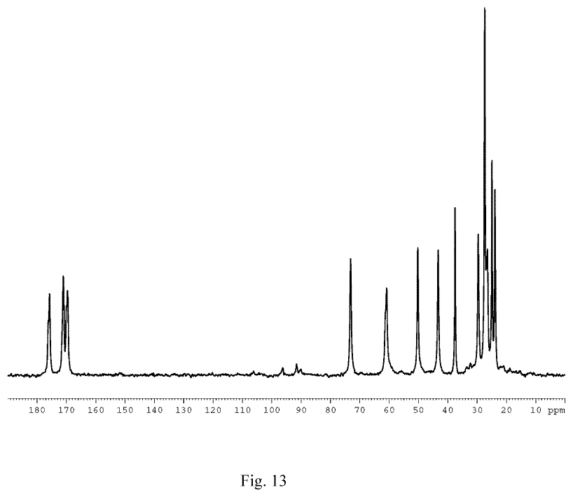

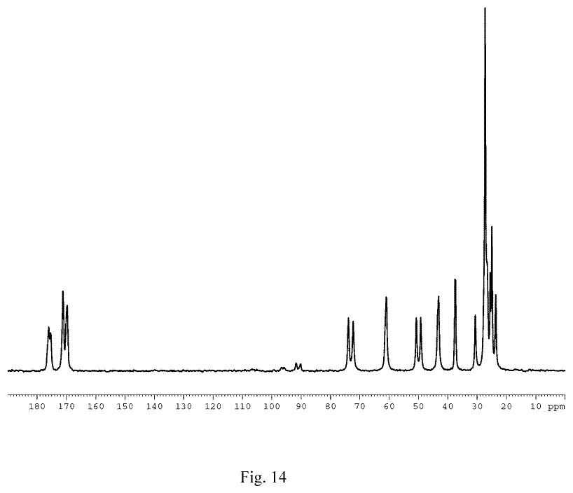

Microscopic examinations were performed using an optical microscope or electron microscope. Microscopic examinations of three pure forms of marimastat, as well as the mixture of the forms I and III, revealed that these forms have different crystal morphologies (FIGS. 10, 11 and 12) and different bulk densities, which is particularly important in the formulation preparation process. Generally, the forms I and II have the highest and comparable bulk density.

### Example 35

### Solubility Tests

Solubility tests were performed by dissolving samples of pure forms of marimastat in water. Solubility tests of the crystalline polymorphic forms I, II and III showed that the form III is the fastest soluble in water, followed by the form II, and the form that dissolves the slowest is the form I.

### Example 36

### Analysis of ¹³C CPMAS NMR and ¹⁵N CPMAS NMR Spectra

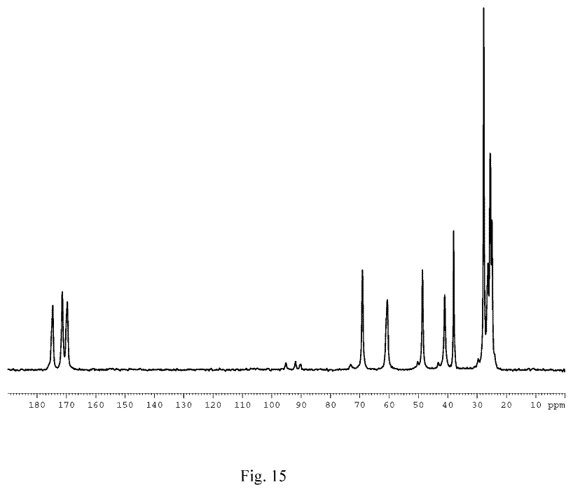

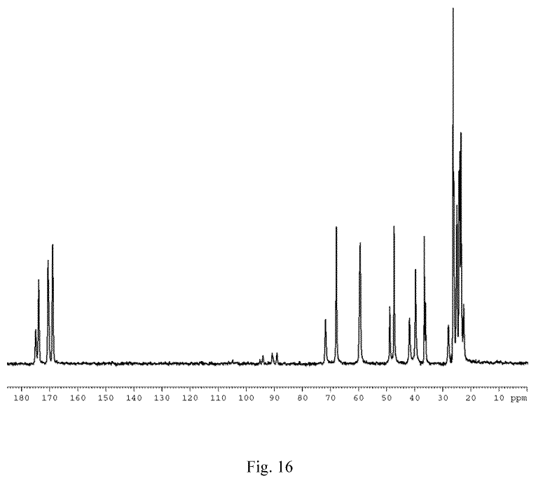

All solid-state spectra were recorded on Bruker Avance III, 600 MHz and Bruker Avance III 400 MHz spectrometers. Samples of the polymorphic forms I, II and III and the mixture of the polymorphic forms I and III were placed in a 4 mm zirconium oxide rotor and spun at a magic angle at 12 kHz (600 MHz) and 8 kHz (400 MHz). A Cross Polarization Magic Angle Spinning (CPMAS) pulse sequence was used to acquire ¹³C and ¹⁵N spectra. The quality of the spectra defined by Full Width at Half Height (FWHH) suggests that each tested sample is an ordered, well-organized material with a high degree of crystallinity. There is no amorphous phase visible in the tested material. Based on the measurements performed, the presence of three polymorphic forms in pure form was demonstrated. NMR analysis of the mixture of the forms I and III was also performed. The ¹³C CPMAS NMR spectra of the forms I, II and III and the mixture of the forms I and III are shown in FIGS. 13, 14, 15 and 16.

### The Polymorphic Form I

¹³C NMR (solid state), δ_{(C—O)}=175.7 ppm, δ_{(C—O)}=171.1 ppm, δ_{(C—O)}=169.8 ppm, δ_{(C—H)}=73.1 ppm, δ_{(C—H)}=61.0 ppm, δ_{(C—H)}=50.3 ppm, δ_{(C—H)}=43.3 ppm, δ_{(Cquat)}=37.5 ppm, δ(CH₂)=29.6 ppm,
δ_{(C—H3)}=27.4 ppm, δ_{(C—H)}=26.9 ppm, δ_{(C—H3)}=26.5 ppm, δ_{(C—H3)}=24.9 ppm, δ_{(C—H3)}=23.9 ppm.

¹⁵N NMR (solid state), 8=168.4 ppm, 8=117.2 ppm, 8=116.5 ppm.

### The Polymorphic Form II

¹³C NMR (solid state), δ_{(C—O)}=176.0 ppm, δ_{(C—O)}=175.3 ppm, δ_{(C—O)}=171.1 ppm, δ_{(C—O)}=169.7 ppm, δ_{(C—H)}=73.9 ppm, δ_{(C—H)}=72.3 ppm, δ_{(C—H)}=61.0 ppm, δ_{(C—H)}=50.7 ppm, δ_{(C—H)}=49.2 ppm,
δ_{(C—H)}=43.3 ppm, δ_{(C—H2)}=37.6 ppm, δ_{(Cquat)}=37.4 ppm, δ(CH₂)=30.6 ppm, δ_{(C—H3)}=27.2 ppm,
δ_{(C—H)}=26.6 ppm, δ_{(C—H3)}=25.5 ppm, δ_{(C—H3)}=25.0 ppm, δ_{(C—H3)}=23.6 ppm.

¹⁵N NMR (solid state), 8=168.3 ppm, 8=117.1 ppm, 8=116.5 ppm.

### The Polymorphic Form III

¹³C NMR (solid state), δ_{(C—O)}=174.6 ppm, δ_{(C—O)}=171.4 ppm, δ_{(C—O)}=169.6 ppm, δ_{(C—H)}=69.2 ppm,
δ_{(C—H)}=60.6 ppm, δ_{(C—H)}=48.6 ppm, δ_{(C—H)}=41.0 ppm, δ_{(c-quat)}=38.0 ppm, δ_{(C—H2)}=27.8 ppm,
δ_{(C—H3)}=27.8 ppm, δ_{(C—H)}=26.3 ppm, δ_{(C—H3)}=25.6 ppm, δ_{(C—H3)}=25.4 ppm, δ_{(C-H3)}=24.9 ppm.

¹⁵N NMR (solid state), δ=166.6 ppm, δ=117.1 ppm, δ=116.2 ppm.

### The Mixture of the Polymorphic Forms I and III

¹³C NMR (solid state), δ_{(C—O)}=175.7 ppm, δ_{(C—O)}=174.6 ppm, δ_{(C—O)}=171.4 ppm, δ_{(C—O)}=171.1 ppm, δ_{(C—O)}=169.7 ppm, δ_{(C—H)}=73.0 ppm, δ_{(C—H)}=69.1 ppm, δ_{(C—H)}=60.7 ppm, δ_{(C—H)}=50.2 ppm,
δ_{(Cquat)}=48.7 ppm, δ_{(C—H)}=43.2 ppm, δ_{(C—H)}=41.0 ppm, δ_{(Cquat)}=37.9 ppm, δ_{(Cquat)}=37.5 ppm,
δ(CH₂)=29.2 ppm, δ_{(C—H3)}=27.6 ppm, δ_{(C—H3)}=27.3 ppm, δ_{(C—H)}=26.4 ppm, δ_{(C—H)}=26.3 ppm,
δ_{(C—H3)}=25.5 ppm, δ_{(C—H3)}=25.3 ppm, δ_{(C—H3)}=24.8 ppm, δ_{(C—H3)}=23.9 ppm.

¹⁵N NMR (solid state), 8=168.4 ppm, 8=166.6 ppm, 8=117.2 ppm, 8=116.4 ppm.

### Example 37

### Preparation of Tablets Comprising the Mixture of the Crystalline Polymorphic Forms I and III

Tablets comprising the mixture of the crystalline polymorphic forms I and III were produced by wet granulation according to the following steps:
1. Preparation of granulation liquid
2. Granulation
3. Calibration of granulate before drying
4. Drying
5. Calibration of granulate after drying
6. Mixing the granulate with external phase components
7. Tableting

### Step 1: Preparation of Granulation Liquid

A binder is added to water and stirred until completely dissolved.

### Step 2: Granulation

A mixture of internal phase components is granulated with an aqueous solution of the binder obtained in Step 1 in a high-speed granulator.

### Step 3: Calibration of Granulate Before Drying

Before the drying process, the wet granulate is calibrated using a conical mill or oscillating granulator. The wet granulate can also be leveled manually using a steel sieve with an appropriate mesh size.

### Step 4: Drying

The granulate is dried using a fluid bed dryer or tray dryer at a targeted temperature of approximately 55° C. (40-65° C.) until the required weight loss after drying is achieved (0.2-3.0%).

### Step 5: Calibration of Granulate after Drying

The dried granulate is leveled manually, on a steel sieve or using a conical mill or oscillating granulator.

### Step 6: Mixing the Granulate with External Phase Components

All components of the external phase (except a lubricant) are mixed with the granulate in a low-speed mixer, with the speed adjusted to the size of the mixer tank and its filling. The lubricant is added at the end of the mixing stage.

### Step 7: Tableting

Tableting is performed separately for each dose of tablets comprising the mixture of the crystalline polymorphic forms I and III (doses are proportional, obtained by tableting to a specific targeted tablet weight).

Tableting is carried out on a rotary tablet press using appropriate equipment.

### Recipe for Tablets Comprising the Mixture of the Crystalline Polymorphic Forms I and III

The concentration of the mixture of the crystalline polymorphic forms I and III in the product: from 10 to 40%.

Drug doses: proportional, obtained through targeted tablet weights.

TABLE 7
Recipe for tablets comprising the mixture of
the crystalline polymorphic forms I and III

| | Amount per 1 tablet |
|---|---|
| Component | [% w/w] |
| Internal phase (granulate) | |
| mixture of crystalline polymorphic forms I and III (active | 10.0-40.0 |
| substance) | |
| Binder (as a component of granulation liquid)* | 0.5-10.0 |
| Filling agent I | 10.0-50.0 |
| Filling agent II | 5.0-30.0 |
| Disintegrant | 0.0-10.0 |
| Purified water (as a component of granulation liquid)** | q.s. |
| External phase | |
| Filling agent III (type for direct tableting) | 0.0-30.0 |
| Disintegrant | 0.0-10.0 |
| Lubricant | 0.2-5.0 |
| Total | 100.0 |

*Added in the form of a 4-10% aqueous solution
**Removed during the granulate drying process

### REFERENCES

[1] Haq M., Shaeii A. E., Zervos E. E., Rosemurgy A. S.: In vitro and in vivo matrix metalloproteinase production by pancreatic cancer cells and by distant organs. Int. J. Surg. Invest. 2000, 1, 459-465.
[2] Collins H. M., Morris T. M., Watson S. A.: Spectrum of matrix metalloproteinase expression in primary and metastatic colon cancer: relationship to the tissue inhibitors of metalloproteinases and membrane type-1-matrix metalloproteinase. Br. J. Cancer 2001, 84, 1664-1670.
[3] Egeblad M. Werb Z.: New functions for the matrix metalloproteinases In cancer progression. Nat. Rev. Cancer. 2002, 2, 163-176.
[4] Rogowicz A., Zozulińska D., Wierusz-Wysocka B.: Znaczenie metaloproteinaz i ich inhibitorów w progresji naczyniowych powikłań cukrzycy-możliwości terapeutyczne. Pol. Arch. Med. Wewn. 2007,117, 103-108.
[5] Barbara Pijet, Anna Konopka, Emilia Rejmak, Marzena Stefaniuk, Danylo Khomiak, Ewa Bulska, Stanisław Pikul & Leszek Kaczmarek: The matrix metalloproteinase inhibitor marimastat inhibits seizures in a model of kainic acid-induced status epilepticus. Scientific Reports. 2020, 10:21314.
[6] Evans J. D., Stark A., Johnson C. D., Daniel F., Carmichael J., Buckels J., Imrie C. W., Brown P., Neoptolemos J. P.: A phase II trial of marimastat in advanced pancreatic cancer. Br. J. Cancer, 2001; 85:1865-1870.
[7] Jones L. E., Humphreys M. J., Campbell F., Neoptolemos J. P., Boyd M. T.: Comprehensive analysis of matrix metalloproteinase and tissue inhibitor expression in pancreatic cancer: increased expression of matrix metalloproteinase-7 predicts poor survival. Clin. Cancer Res., 2004; 10:2832-2845.
[8] Patten L. C., Berger D. H.: Role of proteases in pancreatic carcinoma. World J. Surg., 2005; 29:258-263.
[9] Henrik S. Rasmussen i Peter P. McCann: Matrix Metalloproteinase Inhibition as a Novel Anticancer Strategy: A Review with Special Focus on Batimastat and Marimastat. Pharmacol. Ther. Vol. 75, No. 1, pp. 69-75, 1997.
Leppert D., Lindberg R. L., Kappos L., Leib S. L.: Matrix metalloproteinases: multifunctional effectors of inflammation in multiple sclerosis and bacterial meningitis. Brain Res. Brain Res. Rev., 2001; 36:249-257.
Yong V. W., Power C., Forsyth P., Edwards D. R.: Metalloproteinases in biology and pathology of the nervous system. Nat. Rev. Neurosci., 2001; 2:502-511.
Richard J Davenport, Robert J Watson: An improved synthesis of the broad spectrum matrix metalloprotease inhibitor marimastat. Tetrahedron Letters, Volume 41, Issue 41, 7.10.2000, pp. 7983-7986.

1. A crystalline polymorphic form of marimastat, which is the crystalline polymorphic form I, characterized by a powder X-ray diffraction pattern obtained by irradiation with Cu Kα radiation, which includes main peaks at 2-Theta° 6.11, 11.10, 12.19, 18.15, 19.57, 20.54, 20.74, 23.35, 24.50 and 26.20±0.2.

2. The crystalline form according to claim 1, characterized in that it is anhydrous.

3. A crystalline polymorphic form of marimastat, which is the crystalline polymorphic form II, characterized by a powder X-ray diffraction pattern obtained by irradiation with Cu Kα radiation, which includes main peaks at 2-Theta° 6.18, 12.26, 14.95, 19.74, 21.53, 24.55, 24.55, 27.73, 30.80 and 37.12±0.2.

4. The crystalline form according to claim 3, characterized in that it is anhydrous.

5. A crystalline polymorphic form of marimastat, which is the crystalline polymorphic form III, characterized by a powder X-ray diffraction pattern obtained by irradiation with Cu Kα radiation, which includes main peaks at 2-Theta° 6.32, 11.05, 12.60, 17.30, 18.36, 20.43, 20.93, 24.05, 26.83 and 40.30±0.2.

6. The crystalline form according to claim 5, characterized in that it is anhydrous.

7. A mixture of the crystalline polymorphic form I of marimastat and the crystalline polymorphic form III of marimastat, characterized by a powder X-ray diffraction pattern obtained by irradiation with Cu Kα radiation, which includes main peaks at 2-Theta° 6.14, 6.30, 11.09, 12.22, 12.60, 18.43, 19.62, 20.51, 20.78 and 20.98±0.2.

8. An amorphous form of marimastat.

9. A process for the preparation of the crystalline polymorphic form I of marimastat defined in claim 1, characterized in that the process includes maceration of an amorphous form of marimastat in a solvent selected from the group comprising 2-methyltetrahydrofuran, isopropyl ether, methyl tert-butyl ether, methylene chloride, ethanol, cyclohexane, 1,2-dichloroethane, trichloroethylene, isopropyl acetate, 2-butanone, pentane and acetonitrile.

10. A process for the preparation of the crystalline polymorphic form I of marimastat defined in claim 1, characterized in that the process includes preparing a saturated solution of marimastat in dimethyl sulfoxide and precipitating the crystals with acetonitrile, ethyl acetate or acetone.

11. A process for the preparation of the crystalline polymorphic form I of marimastat defined in claim 1, characterized in that the process includes crystallization from ethanol.

12. A process for the preparation of the crystalline polymorphic form II of marimastat defined in claim 3, characterized in that the process includes maceration of an amorphous form of marimastat in a solvent selected from chloroform or diethyl ether.

13. A process for the preparation of the crystalline polymorphic form II of marimastat defined in claim 3, characterized in that the process includes the following steps:
a) preparing a saturated solution of marimastat in boiling methanol;
b) adding 2-methyltetrahydrofuran, 1,2-dimethoxyethane or chloroform to the solution;
c) cooling the solution to a temperature of 30-40° C.; and
d) isolating the formed crystals.

14. A process for the preparation of the crystalline polymorphic form II of marimastat defined in claim 3, characterized in that the process includes the following steps:
a) preparing a saturated solution of marimastat in methanol at a temperature of 55-65° C.;
b) mixing the solution with ethyl acetate at a temperature of 55-65° C.;
c) cooling the mixture to room temperature; and
d) isolating the formed crystals.

15. A process for the preparation of the crystalline polymorphic form II of marimastat defined in claim 3, characterized in that the process includes the following steps:
a) preparing a saturated solution of marimastat in dimethyl sulfoxide at a temperature of 40-90° C.;
b) adding chloroform, methylene chloride, 2-methyltetrahydrofuran or 1,2-dimethoxyethane to the solution;
c) cooling the solution to a temperature from room temperature to 40° C.; and
d) isolating the formed crystals.

16. A process for the preparation of the crystalline polymorphic form II of marimastat defined in claim 3, characterized in that the process includes the following steps:
a) preparing a saturated solution of marimastat in dimethyl sulfoxide at a temperature of 50-65° C.;
b) mixing the solution with ethyl acetate at a temperature of 55-65° C.;
c) cooling the mixture to room temperature; and
d) isolating the formed crystals.

17. A process for the preparation of the crystalline polymorphic form II of marimastat defined in claim 3, characterized in that the process includes the following steps:
a) reaction of (R)—N—((S)-3,3-dimethyl-1-methylamino-1-oxobutan-2-yl)-2-((S)-2,2-dimethyl-5-oxo-1,3-dioxolan-4-yl)-4-methylpentanamide with a 50% aqueous solution of hydroxylamine in 2-methyltetrahydrofuran or a mixture of dimethyl sulfoxide and chloroform at room temperature to boiling point;
b) after the reaction is completed, performing azeotropic distillation to remove water; and
c) isolating the formed crystals.

18. A process for the preparation of the crystalline polymorphic form III of marimastat defined in claim 5, characterized in that the process includes preparing a saturated solution of marimastat in methanol, and precipitating the crystals with ethyl acetate.

19. A process for the preparation of the crystalline polymorphic form III of marimastat defined in claim 5, characterized in that the process includes preparing a saturated solution of marimastat in dimethyl sulfoxide and precipitating the crystals with a solution of methanol in water.

20. A process for the preparation of the mixture of the crystalline polymorphic form I of marimastat and the crystalline polymorphic form III of marimastat defined in claim 7, characterized in that the process includes maceration of an amorphous form of marimastat in a solvent selected from the group comprising tetrahydrofuran, 1,2-dimethoxyethane, 2-propanol, 1-propanol, isoamyl alcohol, acetone and heptane.

21. A process for the preparation of the mixture of the crystalline polymorphic form I of marimastat and the crystalline polymorphic form III of marimastat defined in claim 7, characterized in that the process includes preparing a saturated solution of marimastat in methanol and precipitating the crystals with acetonitrile, acetone, isopropyl acetate or 2-methyltetrahydrofuran.

22. A process for the preparation of the mixture of the crystalline polymorphic form I of marimastat and the crystalline polymorphic form III of marimastat defined in claim 7, characterized in that the process includes crystallization from a boiling solvent selected from ethanol, n-propanol or isopropanol.

23. A process for the preparation of the mixture of the crystalline polymorphic form I of marimastat and the crystalline polymorphic form III of marimastat defined in claim 7, characterized in that the process includes preparing a saturated solution of marimastat in boiling ethanol and precipitating the crystals with ethyl acetate.

24. A process for the preparation of the mixture of the crystalline polymorphic form I of marimastat and the crystalline polymorphic form III of marimastat defined in claim 7, characterized in that the process includes preparing a saturated solution of marimastat in dimethylformamide and precipitating the crystals with isopropyl acetate.

25. A pharmaceutical composition characterized in that it comprises the crystalline polymorphic form of marimastat defined in claim 1 and a pharmaceutically acceptable carrier.

26. A pharmaceutical composition characterized in that it comprises the crystalline polymorphic form of marimastat defined in claim 3 and a pharmaceutically acceptable carrier.

27. A pharmaceutical composition characterized in that it comprises the crystalline polymorphic form of marimastat defined in claim 5 and a pharmaceutically acceptable carrier.

28. A pharmaceutical composition characterized in that it comprises the mixture of the crystalline polymorphic form I of marimastat and the crystalline polymorphic form III of marimastat defined in claim 7 and a pharmaceutically acceptable carrier.

29. A pharmaceutical composition characterized in that it comprises the amorphous form of marimastat defined in claim 8 and a pharmaceutically acceptable carrier.

30. The composition according to any one of claims 25-29, characterized in that it is in the form of a tablet, modified-release tablet, pill, capsule, powder, granules, pellets, suspension, emulsion, solution, oral liquid forms, e.g. syrup, solution or suspension for injection, solution for infusion, eye drops, ointment, gel, suppository, globule or therapeutic system, e.g. implant, vaginal ring, nanofiber.

31. The pharmaceutical composition defined in any one of claims 25-29 for use as a medicament.

32. The pharmaceutical composition defined in any one of claims 25-29 for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

33. The crystalline polymorphic form of marimastat defined in claim 1 for use as a medicament.

34. The crystalline polymorphic form of marimastat defined in claim 3 for use as a medicament.

35. The crystalline polymorphic form of marimastat defined in claim 5 for use as a medicament.

36. The mixture of the crystalline polymorphic form I of marimastat and the crystalline polymorphic form III of marimastat defined in claim 7 for use as a medicament.

37. The amorphous form of marimastat defined in claim 8 for use as a medicament.

38. The crystalline polymorphic form of marimastat defined in claim 1 for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

39. The crystalline polymorphic form of marimastat defined in claim 3 for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

40. The crystalline polymorphic form of marimastat defined in claim 5 for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

41. The mixture of the crystalline polymorphic form I of marimastat and the crystalline polymorphic form III of marimastat defined in claim 7 for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.

42. The amorphous form of marimastat defined in claim 8 for use in the prevention and treatment of diseases associated with hyperactivity of extracellular matrix metalloproteinases selected from the group comprising post-stroke epilepsy, post-traumatic epilepsy, epilepsy after brain surgery, hypoxic-ischemic encephalopathy, malignant neoplasms, vascular malformations, amyotrophic lateral sclerosis, multiple sclerosis, snake venom poisoning, endometriosis, hemorrhoids, arthritis, nervous system diseases, circulatory system diseases, diabetes and diabetes complications selected from the group comprising diabetic retinopathy and diabetic foot.
